(19) United States
(12) Patent Application Publication
(10) Pub. No.: US 2022/0249326 A1
(43) Pub. Date: Aug. 11, 2022

(54) IN-LINE MEDICATION CRUSHER FOR FEEDING TUBES

(71) Applicant: KIRN MEDICAL DESIGN, LLC, Lexington, KY (US)

(72) Inventors: David S. KIRN, Lexington, KY (US); Kevin J. MAUDSLEY, Lexington, KY (US)

(21) Appl. No.: 17/617,378

(22) PCT Filed: Jun. 15, 2020
(86) PCT No.: PCT/US2020/037746

§ 371 Date: Dec. 8, 2021

Related U.S. Application Data
(60) Provisional application No. 62/860,833, filed on Jun. 13, 2019.

(51) Int. Cl. A61J 7/00; A61M 5/24; A61J 15/00; A61M 5/315

(52) U.S. Cl. CPC A61J 7/0007; A61M 5/2448; A61M 5/31528; A61M 5/31515; A61J 15/0003

(57) ABSTRACT

A syringe for injecting medication into a body includes a barrel having an inner diameter, a plunger for longitudinal movement within the barrel, the plunger having threads along at least a portion thereof for engaging internal threads of a thread ring, and a handle for rotating the plunger relative the barrel. An alternate embodiment of a syringe includes a barrel having internal threads positioned along a first interior wall, a diameter of the internal threads being greater than or equal to a diameter of a second interior wall of the barrel, and a plunger in sealing engagement with the second interior wall having threads for engaging the internal threads of the barrel.

This application claims the benefit of U.S. Provisional Patent Application No. 62/860,833, filed Jun. 13, 2019, the disclosure of which is incorporated herein by reference.

### FIELD OF THE INVENTION

This document relates generally to devices for crushing medication intended for insertion into feeding tubes, and more specifically with a syringe for crushing medication and injecting same into such tubes.

### BACKGROUND OF THE INVENTION

Administration of medications to patients with feeding tubes is a necessary occurrence usually required multiple times per day. Although many medications are available in liquid form, not all are. Therefore, the caregiver must crush certain medications, including pills, into small enough particles that they can pass through the patient's feeding tube. Many feeding tubes are long and have a small inner diameter or lumen so the medications must be thoroughly pulverized.

Typically, the medications are crushed or ground into fragments in a crushing device. The crushed fragments are then transferred to a cup and mixed with a liquid. A syringe is used to draw up the mixture and inject it into the feeding tube. This process requires multiple parts including a crusher, a container for the medication to be crushed in or moved into, a cup and a syringe. Further, some of the medication fragments are typically lost in the process as they are transferred between the crusher, the cup, and the syringe. This can result in under-dosing of the patient.

Accordingly, a device is needed which can crush the medication and facilitate mixing the crushed fragments with a liquid and directly injecting the mixture into a feeding tube.

### SUMMARY OF THE INVENTION

In accordance with the purposes and benefits described herein, a syringe for crushing and injecting medication into a body is provided. The syringe includes a barrel having an inner diameter, a plunger for longitudinal movement within the barrel, the plunger having threads along at least a portion thereof for engaging internal threads of a thread ring, and a handle for rotating the plunger relative the barrel.

In another embodiment, the plunger sealingly engages the inner diameter of the barrel. In still another embodiment, the syringe further includes a gasket supported by the plunger for sealingly engaging the inner diameter of the barrel. In yet still another embodiment, the gasket is supported within a recess formed in the plunger. In one other embodiment, the recess is formed proximal a distal end of the plunger.

In still another embodiment, a surface of a distal end of the plunger is hard. In yet another, the hard surface of the plunger engages an interior surface of the barrel to crush the medication. In still yet another, the interior surface of the barrel is at least one of spherical, pyramidal, or frustroconical shaped. In one other embodiment, the interior surface of the barrel includes at least one of ridges, scallops, and creases, and/or is abraded. In another embodiment, the hard surface of the plunger is at least one of spherical, pyramidal, and frustroconical shaped, and in yet another, the hard surface of the plunger includes at least one of ridges, scallops, and creases, and/or is abraded. In another embodiment, the interior distal surface is a tapering frustroconical shaped wall having radially directed creases.

In one other embodiment, the interior distal surface of the barrel defines an aperture through which the medication is injected into the body. In another embodiment, the hard surface of the plunger is generally frustroconical shaped. In still another embodiment, the hard surface of the plunger is inwardly tapering and includes radially directed ridges extending from the surface. In another, a protuberance extends from the hard surface of the plunger.

In another embodiment, the plunger includes a tip. In still another embodiment, the hard surface of the distal end of the plunger is on the tip. In yet another, the gasket is supported by the tip. In still yet another, the recess supporting the gasket is formed in the tip.

In yet still another embodiment, the tip is detachable.

In one other embodiment, the plunger defines a first receptacle for receiving a lug attached to the tip and shaped to prevent rotation of the tip relative the plunger. In yet another embodiment, the first receptacle is polygonal shaped. In still another, the first receptacle is hexagonally shaped and the lug is correspondingly hexagonally shaped.

In another embodiment, the syringe includes at least two locking tabs extending from the tip. In still another embodiment, the locking tabs engage a wall defining a proximal portion of the first receptacle to secure the tip to the plunger. In one other embodiment, the plunger includes a second receptacle and the locking tabs engage a wall defining a distal portion of the second receptacle to secure the tip to the plunger. In still another, the at least two locking tabs are flexible. In another embodiment still, each of the at least two locking tabs have a tapered surface for engaging an aperture defined by a wall, wherein the engagement compresses the at least two locking tabs from a normal position to a compressed position. In one other embodiment, the at least two locking tabs are biased to the normal position. In still yet another embodiment, the at least locking tabs include barbs to prevent removal of the tip from the plunger. In another embodiment, the at least two locking tabs return to the normal position or an intermediate position, from the compressed position within the aperture, once the tapered surface and barbs are positioned on a distal side of the wall.

In another embodiment, the syringe includes an ejector. In still another embodiment, the ejector extends through the handle and partially through the plunger. In yet another embodiment, the ejector further extends through a support ring supported attached to the plunger. In another embodiment, the ejector is biased to an extended position such that a portion of the ejector extends beyond a distal end of the handle for actuation by a user.

In another embodiment, the syringe includes a spring which biases the ejector to the extended position, wherein a first end of the spring is positioned within the second receptacle defined by the plunger and a second end of the spring is supported by the ejector. In yet another embodiment, a tip of the ejector is shaped to engage the tapered surfaces of the at least two locking tabs and move the at least two locking tabs from the normal position or intermediate position to a compressed position. In still another embodiment, the ejector extends through the handle and partially through the plunger. In another still, the ejector further extends through a support ring supported within the plunger. In still yet another embodiment, the ejector is biased to an extended position such that a portion of the ejector extends beyond the handle for actuation by a user.

In another embodiment, the syringe includes a spring which biases the ejector to the extended position, wherein a first end of the spring is positioned within the second receptacle of the plunger and a second end of the spring is supported by a tip of the ejector. In yet another embodiment, the tip of the ejector is further shaped to receive at least a portion of the tapered surfaces of the at least two locking tabs.

In yet another embodiment, the thread ring includes at least two arms. In another embodiment, the at least two arms include two opposing arms. In still another embodiment, the at least two arms extend along an exterior surface of the thread ring. In yet another embodiment, each of the at least two arms includes a latch for engaging one or more lugs extending from the barrel. In still yet another embodiment, the latches are sized to pass through one or more gaps between the one or more lugs during removal of the barrel.

In one other embodiment, the threads of the plunger extend partially around the plunger. In The syringe for crushing and injecting medication into a body of claim 49, wherein the threads of the plunger are retractable.

In still another embodiment, the syringe includes a hinge attached to the plunger and attached to the threads. In another embodiment, the hinge is a living hinge. In still another, the living hinge and threads are biased to a retracted position.

In another embodiment, the syringe includes a cam attached to the handle for selectively engaging the threads for movement between a retracted position and an extended position sufficient to engage the internal threads of the thread ring. In yet another, the syringe further includes a neck attached to the handle and the cam. In one other embodiment, the handle, neck, and cam are integrally formed.

In yet another embodiment, the syringe includes a hinge supported by the plunger and attached to the threads. In still yet another, the hinge is a living hinge integrally formed with the plunger. In one other embodiment, a ring defining a ledge is attached to a proximal end of the plunger, and wherein the handle includes at least one latch for engaging the ledge. In another, the ring is integrally molded.

In still another embodiment, a ledge is formed in the plunger, and wherein the handle includes at least one latch for engaging the ledge. In another embodiment, rotation of the cam is limited between a first position wherein the retractable threads are in the retracted position and a second position wherein the retractable threads are in the extended position.

In one other embodiment, the syringe includes a cam extending from the handle for selectively engaging the plunger threads, wherein rotation of the cam is limited between a first position wherein the plunger threads are in a retracted position and a second position wherein the plunger threads are in an extended position. In another, the at least one latch of the handle includes a deflectable arm. In still another embodiment, the at least one latch is flexible and extends from the handle. In yet still another embodiment, the at least one latch of the handle has a tapered surface for engaging the ring, wherein the engagement deflects the at least one latch from a normal position to a deflected position. In one other embodiment, the at least one latch includes a barb to secure the handle to the plunger. In another, the at least one latch returns to the normal position or an intermediate position between the extended and normal positions once the tapered surface and barb is positioned distally of the ledge. In still another, the barb engages the ledge and holds the cam in position within the plunger while allowing relative rotation between the plunger and the handle.

In an additional embodiment, the ejector extends through the handle, the cam, the support ring and at least partially through the plunger. In still another embodiment, the handle, the cam, and the plunger rotate at least partially around the ejector. In yet another, the cam includes first and second lobes. In one other embodiment, a stop extends from one of the first and second lobes. In still yet another embodiment, the support ring is attached to a rib extending linearly along an inner wall of the plunger. In another embodiment, the support ring and rib are integrally molded with the plunger. In still another, the stop engages the rib to limit rotation of the cam. In another embodiment, the handle includes a plurality of grips.

In accordance with another aspect of the invention, a syringe for crushing and injecting medication into a body is provided. The syringe includes a barrel having internal threads positioned along a first interior wall, a diameter of the internal threads being greater than or equal to a diameter of a second interior wall of the barrel, and a plunger in sealing engagement with the second interior wall having threads for engaging the internal threads of the barrel.

In another embodiment, a surface of a distal end of the plunger is hard. In yet another embodiment, the surface of the distal end of the plunger is pyramidal or frustroconical in shape. In another still, the surface of the distal end of the plunger includes at least one ridge, scallop, and crease, and/or is abraded.

In one other embodiment, a gasket supported by the plunger for engaging the second interior wall of the barrel. In still another embodiment, the gasket is supported within a recess formed in the plunger.

In still another embodiment, the threads of the plunger extend partially around the plunger. In yet still another, the threads of the plunger are retractable.

In another embodiment, the syringe includes a hinge supported by the plunger and attached to the plunger threads. In one other embodiment, the hinge is integrally formed with the plunger.

In still another embodiment, the syringe includes a cam positioned within the plunger for selectively engaging the plunger threads for movement between a retracted position and an extended position sufficient to engage the internal threads of the barrel.

In yet still another embodiment, the syringe includes a cam positioned within the plunger for selectively engaging the plunger threads for movement between a retracted position and an extended position engaging the internal threads of the barrel.

In another embodiment, the syringe includes a handle supported by the plunger for at least rotating the plunger. In still another embodiment, a ring is attached to a proximal end of the plunger forming a ledge, and wherein the handle includes at least one latch for engaging the ledge. In yet another, the ring and plunger are integrally molded. In another embodiment, a ledge is formed in the plunger, and wherein the handle includes at least one latch for engaging the ledge.

In yet another embodiment, the plunger includes a hole for receiving an axle extending from the cam.

In another embodiment, the cam includes a stop for engaging the plunger to limit rotation of the cam. In still another embodiment, rotation of the cam is limited between a first position wherein the retractable threads are in a non-extended position and a second position wherein the retractable threads are in an extended position. In yet still another embodiment, at least two grips extend from a distal portion of the barrel. In another, the handle and the cam are integrally molded.

In another aspect of the invention, a syringe for crushing and injecting medication into a body includes a barrel, a plunger for longitudinal movement within the barrel, the plunger having threads along at least a portion thereof for engaging internal threads of a component, and a handle for rotating the plunger relative the barrel.

In one other embodiment, the component is a thread ring.

In still another embodiment, the barrel includes a first portion, and a second portion including the component.

In yet still another embodiment, the first portion of the barrel has an inner diameter lesser than or equal to an inner diameter of the internal threads of the component.

In yet one other embodiment, the internal threads of the component are positioned along a first interior wall of the barrel, and a diameter of the internal threads of the component is greater than or equal to a diameter of a second interior wall of the barrel.

In still another aspect of the invention, a syringe for crushing and injecting medication into a body includes a barrel having an inner diameter, a plunger for longitudinal movement within the barrel, the plunger having retractable threads along at least a portion thereof for selectively engaging internal threads of a thread ring and a tip, a handle for rotating the plunger relative the barrel, and an ejector extending beyond the handle and at least partially through the plunger for engaging the tip for removal.

In one other embodiment, a surface of the tip is hard. In still another embodiment, the hard surface of the tip engages an interior surface of the barrel to crush the medication. In yet still another embodiment, the hard surface of the tip is at least one of spherical, pyramidal, or frustroconical in shape, includes at least one ridge or scallop, and/or is abraded. In another, the hard surface of the tip includes at least one of ridges, scallops, and creases, and/or is abraded.

In another embodiment, a gasket for sealingly engaging the inner diameter of the barrel is supported in a recess formed in the tip. In still another embodiment, the tip includes a lug and the plunger defines a receptacle for receiving the lug to prevent rotation of the tip relative the plunger.

In yet still another embodiment, the syringe further includes at least two locking tabs extending from the tip. In another embodiment, each of the at least two locking tabs have a tapered surface for engaging an aperture defined by a wall attached to the plunger, wherein the engagement compresses the at least two locking tabs from a normal position to a compressed position. In one other embodiment, the at least two locking tabs are biased to the normal position. In still yet another embodiment, the at least two locking tabs return to the normal position or an intermediate position, from the compressed position, when the lug is positioned in the receptacle.

In one other embodiment, the syringe further a cam attached to the handle for selectively engaging the retractable threads for movement between a retracted position and an extended position.

In accordance with another aspect of the invention, a syringe for crushing and injecting medication into a body includes a barrel having internal threads positioned along a first interior wall, a diameter of the internal threads being greater than or equal to a diameter of a second interior wall of the barrel, a plunger for longitudinal movement within the barrel, the plunger having retractable threads along at least a portion thereof for selectively engaging the internal threads of the barrel and a tip, and a handle for rotating the plunger relative the barrel.

In another embodiment, the syringe further includes a cam attached to the handle for selectively engaging the retractable threads for movement between a retracted position and an extended position. In still another, a surface of the tip is hard. In another yet, the hard surface of the tip engages an interior surface of the barrel to crush the medication. In still yet another embodiment, the hard surface of the tip is at least one of spherical, pyramidal, or frustroconical shaped, includes at least one of a ridge, a scallop, and/or a crease, and/or is abraded. In another embodiment, the hard surface of the tip includes at least one of ridges, scallops, and creases, and/or is abraded. In yet another embodiment, a gasket for sealingly engaging the inner diameter of the barrel is supported in a recess formed in the plunger.

In another aspect of the invention, a syringe for crushing and injecting medication into a body includes a barrel having an inner diameter, a plunger for longitudinal movement within the barrel, the plunger having retractable threads extending from a living hinge attached to the plunger for engaging internal threads of a thread ring, and a handle for rotating the plunger relative the barrel.

In yet another aspect of the invention, a syringe for crushing and injecting medication into a body includes a barrel having an inner diameter, a plunger for longitudinal movement within the barrel, the plunger having threads along at least a portion thereof for engaging internal threads of a thread ring, a handle for rotating the plunger relative the barrel; and an ejector extending from the handle and at least partially through the plunger for engaging a tip for removal.

In still yet another aspect of the invention, a syringe for crushing and injecting medication into a body includes a barrel having an inner diameter, a plunger for longitudinal movement within the barrel, the plunger having threads along at least a portion thereof for selectively engaging internal threads of a thread ring and a tip supporting a gasket for sealingly engaging the inner diameter of the barrel, and a handle for rotating the plunger relative the barrel.

In another aspect of the invention, a method of providing medication to a body using a syringe includes the steps of occluding a distal opening of a barrel having internal threads, inserting medication into the barrel, inserting a plunger into the barrel a distance sufficient for threads of the plunger to engage the internal threads of the barrel, rotating the plunger in a first direction such that the plunger engages and at least partially crushes the medication, rotating the plunger in a second direction a distance sufficient for threads of the plunger to disengage the internal threads of the barrel, retracting the plunger within the barrel to draw a liquid into the barrel to form a liquid and medication mixture, and injecting the liquid and medication mixture into the body.

In yet another possible embodiment, the step of rotating the plunger in a first direction includes the step of rotating a handle attached to the plunger.

In still another possible embodiment, the threads of the plunger are retractable and the step of rotating the plunger in a first direction moves a cam causing the retractable threads to move from a first position wherein the retractable threads are in a normal, non-extended position and a second position wherein the retractable threads are in an extended position.

In accordance with still another aspect of the invention, a method of providing medication to a body using a syringe includes the steps of: (a) occluding a distal opening of a barrel; (b) inserting medication into the barrel; (c) attaching the barrel to the syringe; (d) rotating a handle attached to a cam in a first direction to push retractable threads of a plunger outward sufficient for plunger threads to engage internal threads of a thread ring; (e) further rotating the handle in the first direction such that the plunger engages and at least partially crushes the medication against a surface of the barrel; (f) rotating the handle in a second direction a distance sufficient for the plunger threads to disengage the internal threads of the thread ring; (g) retracting the plunger within the barrel to draw a liquid into the barrel to form a liquid and medication mixture; and (h) injecting the liquid and medication mixture out of the barrel into the body.

In another possible embodiment, the plunger includes a removeable tip. In still another possible embodiment, the removeable tip includes a hard surface for engaging and at least partially crushing the medication.

In yet another possible embodiment, the plunger threads are retractable.

In one other possible embodiment, the step of rotating the handle in a first direction causes the cam to engage the retractable threads and to move the retractable threads from a normal, non-extended position to an extended position.

In another possible embodiment, the method further includes the step of rotating the barrel relative the thread ring to facilitate removal of the barrel.

In still yet another possible embodiment, the method further includes the step of applying pressure to an end of an ejector extending from the handle sufficient to release the tip from the plunger.

In the following description, there are shown and described several embodiments of a syringe for crushing and injecting medication into a body and related methods of providing medication to a body using a syringe. As it should be realized, the methods and described syringes are capable of other, different embodiments and their several details are capable of modification in various, obvious aspects all without departing from the methods and syringes as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate several aspects of the invention and together with the description serve to explain certain principles thereof. In the drawing figures:

FIG. 1 is a perspective, partially exploded view of a syringe for crushing and injecting medication directly into a patient;

FIG. 2 is a perspective top view of an interior surface of a barrel of the syringe;

FIG. 3 is a cross section plan view of a tip of a plunger of the syringe;

FIG. 4 is cross section plan view of a plunger of the syringe including a tip, retractable threads, and an ejector;

FIG. 5 is a perspective view of the syringe;

FIG. 6 is a perspective, partially exploded view of the syringe illustrating a plunger, a thread ring, and a handle;

FIG. 7 is cross section plan view of a plunger of the syringe including retractable threads, an ejector, a cam, and a handle;

FIG. 8 is a perspective cross section plan view of a plunger of the syringe including retractable threads, an ejector, a handled, a cam, and a stop extending from the cam;

FIG. 9A is an end view of a plunger showing a spherical surface;

FIG. 9B is an end view of the plunger showing a frustoconical surface with ridges and/or scallops;

FIG. 9C is an end view of the plunger showing a pyramidical surface;

FIG. 9D is an end view of the plunger showing a simple frustoconical surface without ridges and/or scallops;

FIG. 10 is a perspective, partially exploded view of another embodiment of a syringe for crushing and injecting medication directly into a patient; and

FIG. 11 is a different perspective, partially exploded view of the other embodiment of the syringe for crushing and injecting medication directly into a patient.

Reference will now be made in detail to the present described embodiments of the invention, examples of which are illustrated in the accompanying drawing figures, wherein like numerals are used to represent like elements.

### DETAILED DESCRIPTION

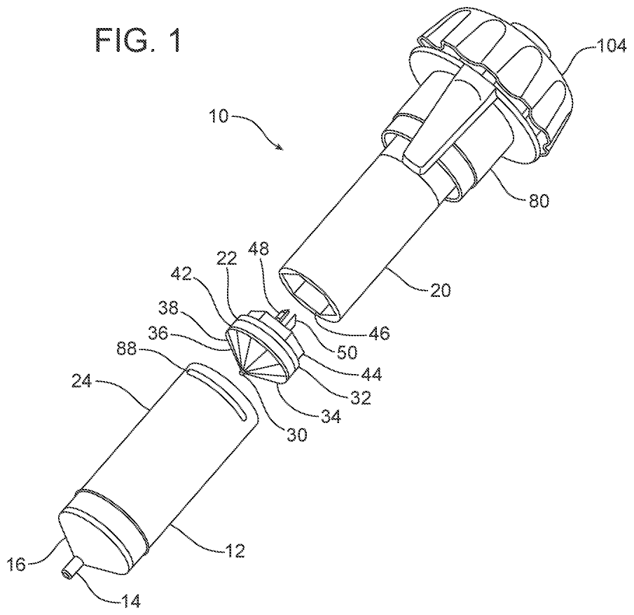

Reference is now made to FIG. 1 which illustrates a syringe 10 for crushing medication in a partially exploded view. In this embodiment, the syringe 10 is designed to allow a user to crush one or more medications (in pill form or otherwise) within a chamber defined by a barrel, draw a mixing fluid into the chamber, and inject the mixture or content of the chamber into a body/patient (e.g., through a feeding or other type of tube) (not shown). Advantageously, the crushed medication, whether in fragmented or powder form, remains within the chamber throughout the process until the crushed medication is directly transferred into the body.

In the described embodiment, the syringe 10 includes an outer barrel 12 having a connector 14 centrally positioned at a distal end 16 of the barrel. The connector 14 is capable of mating with similar connectors, for example, on feeding tubes. The connector 14 may be a slip tip, as shown, or any sort of industry standard fitting such as connections and/or catheter tips sold under the EnFit® or Luerlok® brands or others so long as the feeding tube or the like includes a mating connector/receptacle.

As shown, the barrel 12 is generally cylindrical in shape. An interior wall 18 of the barrel 12 defines a substantially consistent inner diameter throughout a majority of its length. A plunger 20 supporting a gasket 22 or O-ring sealingly engages the interior wall 18 of the barrel 12. As is known in the art, the inner diameter of the barrel 12 is slightly larger than an outside diameter of the plunger gasket 22 (or may be substantially equal to in other embodiments) so that the plunger 20 may pass through the barrel in sealing engagement therewith. This sealing engagement functions the same as a medical syringe allowing the plunger 20 to force the content of the chamber through an opening at the connector 14.

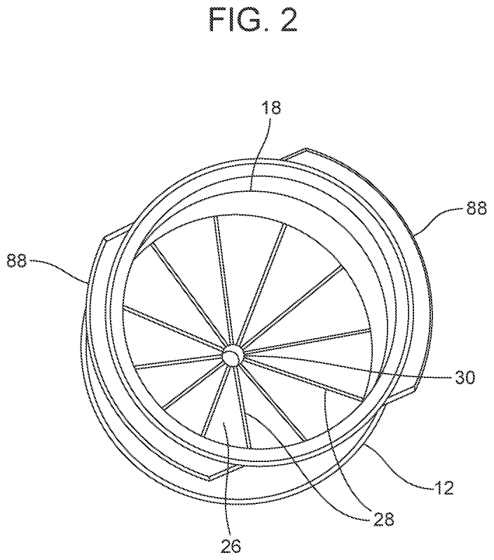

As shown in FIG. 2, the interior wall 18 of the barrel 12 has a substantially consistent inner diameter throughout a majority of its length and transitions into an interior distal surface 26 as it approaches the distal end 16. The interior distal surface 26 is shaped to facilitate the crushing of medication(s). In the described embodiment, the interior distal surface 26 is a downwardly tapering frustoconical wall with radially directed creases 28. The downwardly tapering frustroconical wall 26 terminates at a flat, circular bottom wall 30 which defines an aperture through which the content exits the barrel 12. The frustoconical wall 26 and radially directed creases 28 optimize crushing of the medication(s). In other words, the interior distal surface 26 serves as the mortar, for example, during the crushing phase of the method as described below. It should be noted, however, that the interior distal surface 26 may take other shapes or forms sufficient to facilitate the crushing of medication(s). For example, the interior distal surface 26 may be abraded, include ridges, creases, scallops, and/or be spherical and/or pyramidical in shape.

Returning to FIG. 1, the plunger 20 includes a tip 32. An exterior surface 34 is located at a distal end of the tip 32 and generally abuts the interior distal surface 26 of the barrel 12 when the plunger 20 is fully inserted. The surface 34 must be hard enough to crush the medication(s) (e.g., to serve as a pestle during the crushing phase of the method). In the described embodiment, the exterior surface 34 has a frustoconical shape. It should be noted, however, that the exterior surface 34 may take other shapes or forms sufficient to facilitate the crushing of medication(s). In addition, the exterior surface 34 may be described as including an inwardly tapering surface 36 from which radially directed ridges 38 extend. The radially directed creases 28 and radially directed ridges 38 provide grinding surfaces used to crush the medication(s) therebetween when rotated.

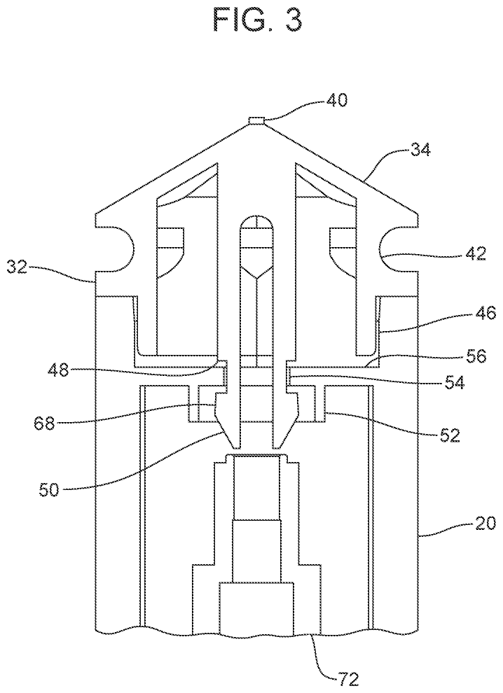

The exterior surface 34 may terminate at a protuberance or nipple 40, in the described embodiment, which is centrally positioned at a distal end. The protuberance 40 extends away from the exterior surface 34, generally aligns with the aperture defined by the bottom wall 30 of the barrel 12, and functions to clear the aperture of crushed medication(s). Proximal to the exterior surface 34, a recess, groove, or channel 42 is formed in the tip 32 for receiving the gasket 22, O-ring, or other type of gasket. The channel 42 is best shown in FIG. 3. As noted above, the gasket 22 provides a fluid tight seal for a mixing liquid and prevents crushed medication fragments from escaping throughout the process.

As further shown in FIG. 1, the barrel 12 and tip 32 are detachable in the described embodiment and may be discarded after use and replaced for future use(s). In other embodiments, the entire syringe 10 may be discarded after use and the tip 32 may be attached or integrally molded with the plunger 20 in such syringes. In order to prevent rotation of the detachable tip 32, a polygonal shaped receptacle 46 is formed in a distal end portion of the plunger 20 for receiving a proximal portion 44 of the tip having a corresponding polygonal lug. In the described embodiment, the receptacle/lug is hexagonal but other polygons or any shape sufficient to prevent rotation of the tip 32 relative the plunger 20 may be utilized.

A pair of locking tabs 48 extend centrally from the tip beyond a proximal end of the tip for securing the tip to the plunger 20 as shown in FIG. 3. The locking tabs 48 are flexible and include a tapered surface 50 along a proximal portion of each tab which acts as bearing surface during insertion into a receptacle 52 of the plunger 20. The locking tabs 48 are biased to a normal position wherein a diameter of a circle drawn around the outsides of the locking tabs is larger than a diameter of an aperture 54 defined by a wall 56. In the described embodiment, the wall 56 forms a proximal portion of the receptacle 46 and a distal portion of the receptacle 52. In this manner, the flexible locking tabs 48 are compressed through contact with the wall 56 upon insertion of the tip 32 into the aperture 54.

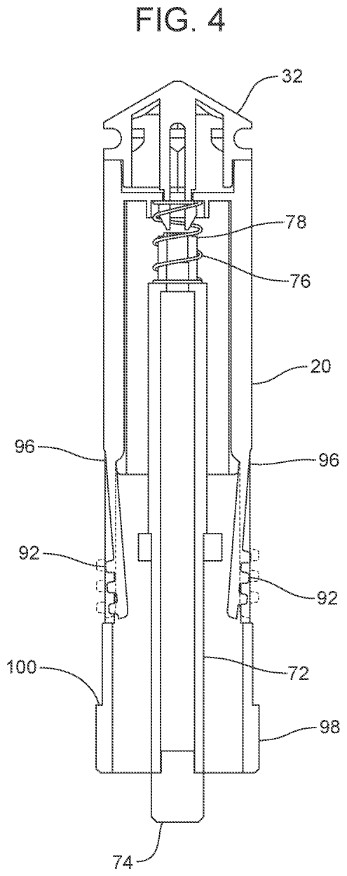

Once the tip 32 is seated in the polygonal shaped receptacle 46 of the syringe 20, the proximal portions 68 of the locking tabs 48 extend through the aperture of the wall 56 a distance sufficient to allow the locking tabs to return from the compressed position attained during insertion toward the normal position. In the present embodiment, the locking tabs 48 are precluded by the size of the aperture of the wall 56 from reaching the normal position. Other embodiments may function differently in this regard, for example, the aperture may be sized to allow the locking tabs 48 to return to the normal position. In this intermediate position, barbs 70 formed on the proximal portion of each locking tab 48 extend beyond the aperture and preclude removal of the tip 32. In other words, the barbs 70 of the locking tabs 48 hold the tip 32 in place within the plunger 20 through contact with the wall 56. Removal of the tip 32 for disposal is facilitated using an ejector 72 extending centrally within the plunger 20 as shown in FIGS. 3 and 4.

More specifically, the ejector 72 may be moved toward the tip 32 by pressing on an end 74. As shown, ejector 72 is biased to an extended position by a spring 76. A first end of the spring is positioned within the receptacle 52 defined by the plunger 20 and a second end of the spring is supported by a tip 78 of the ejector 72. Using a thumb, other digit, or other means, the user applies pressure to the ejector end 74 sufficient to overcome the bias of the spring 76. In this manner, the ejector 72 moves toward the tip 32 and engages the locking tabs 48. The tip 78 of the ejector 72 is shaped to contact the tapered surfaces 50 of each locking tab 48, to receive at least a portion of the end portions 68 of the locking tabs 48, and to move the locking tabs 48 from the normal or intermediate position wherein the tip 32 is precluded from being removed to the compressed position. In the compressed position, further depression of the ejector 72 pushes the barbs 70 and the end portions 68 of the locking tabs 48 through the aperture 54 releasing the tip 32 for removal. Once the tip is removed, the ejector 72 returns to its biased position and a subsequent tip may be inserted into the polygonal shaped receptacle 46 for further use.

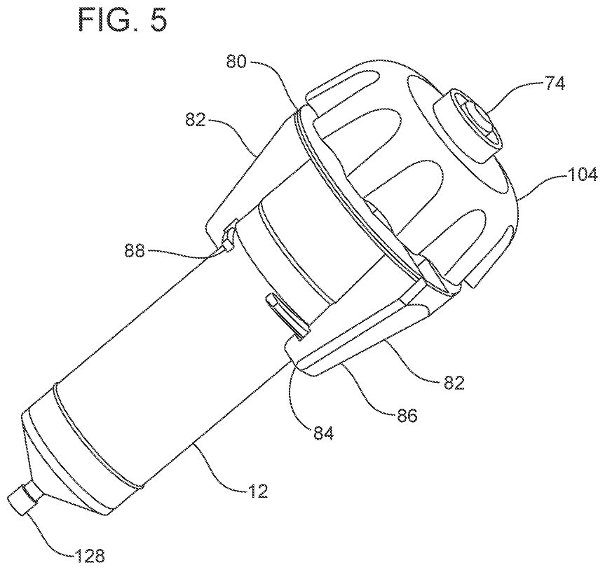

As noted above, the barrel 12 is also detachable and may be discarded after use and replaced for future use(s). As shown in FIG. 5, the described syringe 10 includes a thread ring 80 for retaining the barrel 12 during use. The thread ring 80 includes arms 82 extending along an exterior surface which allows the user to stabilize the barrel 12 both during a rotational motion or a linear motion as plunger 20 is either rotated or moved in or out of the barrel 12. Although two opposing arms 82 are utilized in the present embodiment, more or fewer arms may be utilized in other embodiments. Each of the arms 82 extend along the thread ring 80 and include a latch 84 formed in a distal portion 86 for engaging one or more lugs 88 extending from the barrel 12. The latches 84 are sized to pass through gaps between the lugs 88 during assembly/insertion of the plunger 20 into the barrel 12. Rotating the barrel 12 or thread ring 80 after assembly/insertion positions the lugs 88 within the latches 84 securing the barrel 12 to the thread ring 80 for use.

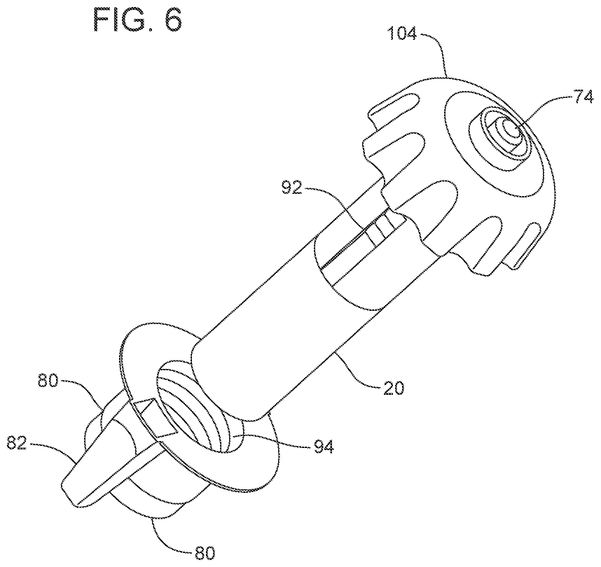

As suggested in FIG. 6, the thread ring 80 also releasably engages retractable threads 92 of the plunger 20. More specifically, the retractable threads 92 releasably engage with threads 94 of the thread ring 80. When engaged, the threads provide a mechanical advantage to generate a substantial force to advance the plunger 20 distally into the barrel 12 to crush the medication(s). In the described embodiment, the retractable threads 92 are integrally molded with the plunger 20 and pivot from a normal or retracted position to an extended position (shown in dash lines in FIG. 4) about a living hinge 96. The living hinge 96 may be located proximally or distally, i.e., at either end of the threads 92. During use, the retractable threads 92 are pushed outward for engagement with the thread ring threads 94 by a cam 112.

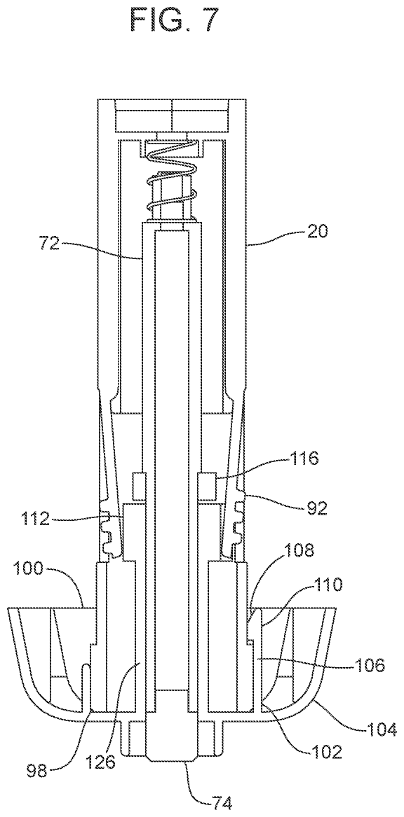

As shown in FIGS. 4 and 7, the plunger 22 includes a ring 98 at a proximal end which defines a ledge 100. One or more latches 102 extending from a handle 104 engage the ring 98 and ledge 100 securing the plunger 20 to the handle 104 while allowing for rotation of the plunger relative to the handle. The ring may be integrally formed with the plunger, as in the described embodiment, or separately formed and attached, may be a partial ring (e.g., lugs), or may take varying shapes so long as a sufficient ledge is created for engaging the one or more latches 106 securing the plunger 20 to the handle 104.

As best shown in FIG. 7, the latch 102 includes a deflectable arm 106 that extends from the handle 104. The latch 102 is flexible and includes a tapered surface 108 along a distal portion 110 which acts as a bearing surface during insertion of the cam 112 into the plunger 20. The latch 102/deflectable arm 106 is biased to a normal position but is deflected outwardly to a deflected position through contact between the tapered surface 108 and the plunger 20 during insertion. Once the cam 112 is seated in the plunger 20, a barb 114 formed on the distal portion 110 of the latch 102 extends a sufficient distance beyond the ring 98 to allow the latch 102 to return from the deflected position to the normal position, or an intermediate position between the extended position and the normal position. In the normal or intermediate position, the barb 114 engages the ring ledge 100 and holds the cam 112 in position within the plunger 20 while allowing relative rotation between the plunger 20 and the cam 112/handle 104. As shown, the ejector 72 extends through the handle 104, into the plunger 20, and through the cam 112 and a support ring 116. The ejector 72 acts as an axle around which the handle 104, the plunger 20, and the cam 112 rotate.

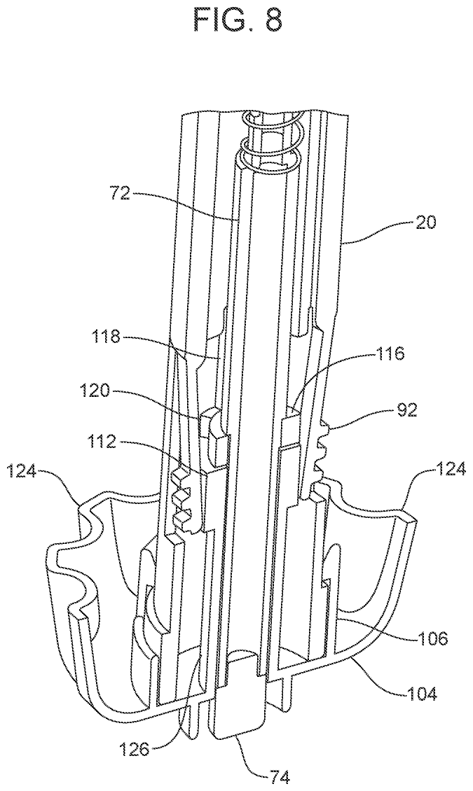

The support ring 116 is attached to a rib 118 extending linearly from and along an inner wall of the plunger 20 as partially shown in FIG. 8. In the described embodiment, the rib 118 and support ring 116 are integrally molded with the plunger 20. The support ring 116 stabilizes the ejector 72 within the plunger 20 and abuts the cam 112 when inserted. The rib 118 further engages a stop 120 extending from one of two lobes 122 of the cam 112 to prevent rotation of the cam beyond 180 degrees. In this manner, initial, clockwise rotation of the cam 112 causes the flexible threads 92 to pivot from their normal position to the extended position where they engage with the thread ring threads 94. At a certain point of rotation, the cam stop 120 engages the rib 118 preventing rotation of the cam 112. Further clockwise rotation of the handle 104 is then translated through the engaged threads and thread ring 80 to the plunger 20 and tip 32. Rotation of the tip 32 serves to grind or crush the medication(s) between the rotating tip surface 34 and the inner surface 26 of the barrel 12. Subsequent rotation of the handle 104 in a counterclockwise direction releases the flexible threads 92 from the threads 94 of the thread ring 80 allowing the plunger 20 to be at least partially withdrawn from the barrel 12 for drawing the mixing fluid into the barrel.

The handle 104 has a large exterior diameter with grips 124 to provide the user with a mechanical advantage as the plunger 20 is screwed into the barrel 12 to crush the medication(s). As best shown in FIG. 5, a diameter of the handle 104 generally corresponds to a diameter of outer edges of the thread ring arms 82. In the described embodiment, a neck 126 to which the cam 112 is secured extends from an underside of the handle 104. The handle 104, neck 126, and cam 112 can be integrally formed or otherwise attached together (e.g., glued fitted or snapped together) so long as there is no rotation between the cam and the handle.

In use, a user removes the barrel 12 from the syringe 10 by rotating the barrel 12 relative the thread ring 80 such that the latches 84 are positioned between barrel lugs 88. In this position, the barrel 12 may be pulled from the syringe 10. Medication to be crushed is placed into the chamber of the barrel 12. A cap or plug 128 may be placed to occlude the distal opening of the barrel 12/connector 14 as needed. The barrel 12 is then reattached to the syringe 10. More specifically, the plunger 20 is reinserted into the barrel 12. The user then rotates the handle 104 clockwise by gripping the thread ring 80 with one hand and the handle 104 with the other hand. The rotation of the handle 104 activates the cam 112 to push the retractable threads 92 outward. At a certain point of rotation, the retractable threads 92 engage with the fixed threads 94 of the thread ring 88, rotation of the cam 112 is stopped, and the plunger 20 is further advanced into the barrel 12 by the screw action. This provides the aforementioned mechanical advantage that generates the substantial force to crush the medication(s).

As described above, the medication(s) is crushed between the exterior surface 34 of the plunger tip 32 and the interior distal surface 26 of the barrel 12 as the plunger is rotated and advanced within the barrel. When the medication is sufficiently crushed, as suggested by a lack of continued rotation, the user rotates the handle 104 counterclockwise causing the cam 112 to disengage the retractable threads 92 allowing them to return to the normal, non-extended position. At this point, the plunger 20 is free to be pulled out of the barrel 12 in a linear manner. If the end cap or plug 128 has been used, the cap may now be removed.

In a next step, the distal fitting 14 of the barrel 12 may be dipped or positioned into a fluid suitable for mixing with the crushed medication(s) and injection into the body. The fluid may be sterile water contained in a cup (not shown) or other fluids with which the crushed medication(s) may be mixed. Once the barrel fitting 14 is positioned in the fluid, the plunger 20 is linearly withdrawn thereby pulling the fluid into the chamber of the barrel 12 where it mixes with the crushed medication(s). The barrel fitting 14 may then be attached to a feeding tube, via a corresponding fitting or otherwise, and the plunger 20 may be advanced to directly inject the mixture of crushed medication(s) and fluid into the feeding tube. If there are medication fragments remaining within the chamber of the barrel 12, additional fluid may be drawn into the chamber, and the injection process repeated.

As noted above, the barrel 12 and tip 32 of the syringe 10 are intended to be used for a single patient so that if any medication fragments remain within the barrel or on the tip, they will not be inadvertently administered to a different patient. In an alternate embodiment, the entire syringe 10 may be intended to be used for a single patient and discarded after use. In order to replace the barrel 12 and tip 32, the user may simply remove the barrel 12 again (as described above). Once removed, the tip 32 may similarly be removed by applying pressure to the ejector end 74 sufficient to overcome the bias of the spring 76. In doing so, the ejector 72 engages the locking tabs 48. More specifically, a tip 78 of the ejector 72 contacts the tapered surfaces 50 of each locking tab 48, receives at least a portion of the end portions 68 of the locking tabs 48, and moves the locking tabs 48 from the normal or intermediate position to the compressed position. In the compressed position, further pressure on the ejector end 74 pushes the barbs 70 and the end portions 68 of the locking tabs 48 through the aperture 54 releasing the tip 32. Once the tip 32 is removed, the ejector 72 returns to its biased position and a subsequent tip may be inserted into the polygonal shaped receptacle 46 for further use. A subsequent barrel may be positioned on the syringe 10 at this point or after subsequent medication(s) to be crushed is placed into the chamber of the subsequent barrel.

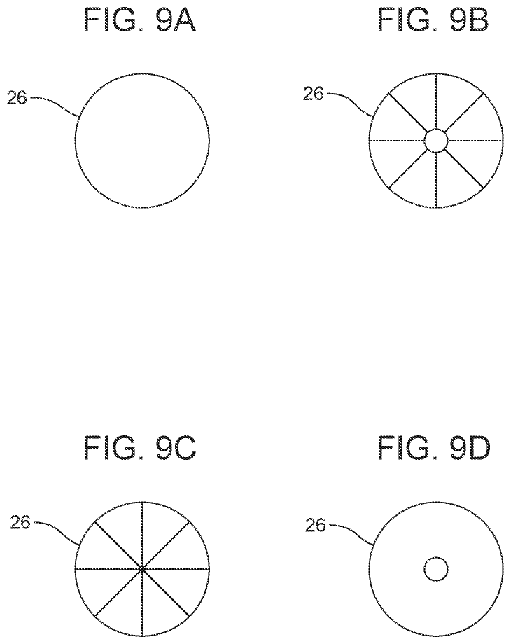

The foregoing has been presented for purposes of illustration and description. It is not intended to be exhaustive or to limit the embodiments to the precise form disclosed. Obvious modifications and variations are possible in light of the above teachings. In additional embodiments, for example, a shape of the interior distal surface 26 may be spherical (see FIG. 9A), frustoconical with ridges and/or scallops (see FIG. 9B), pyramidical (see FIG. 9C), or simply frustoconical without ridges and/or scallops (see FIG. 9D). In essence, the interior distal surface 26 may take any shape so long as the surface is sufficient to crush medication.

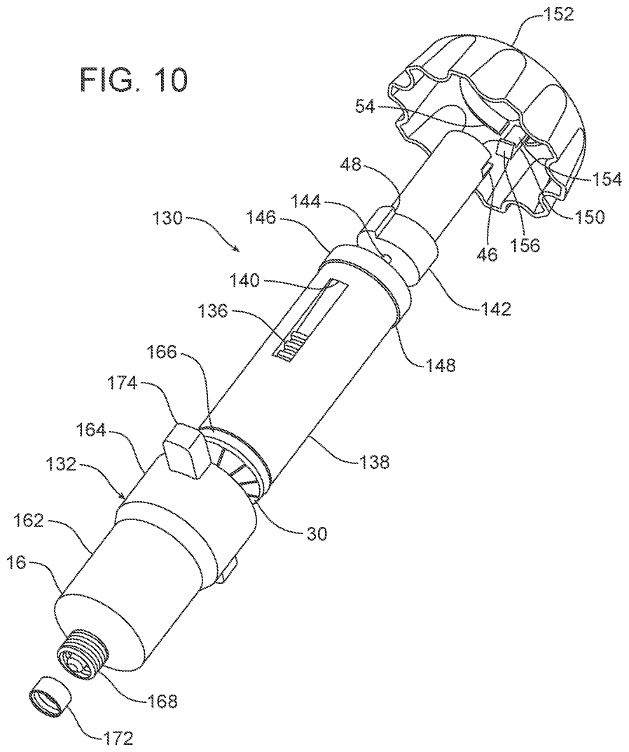

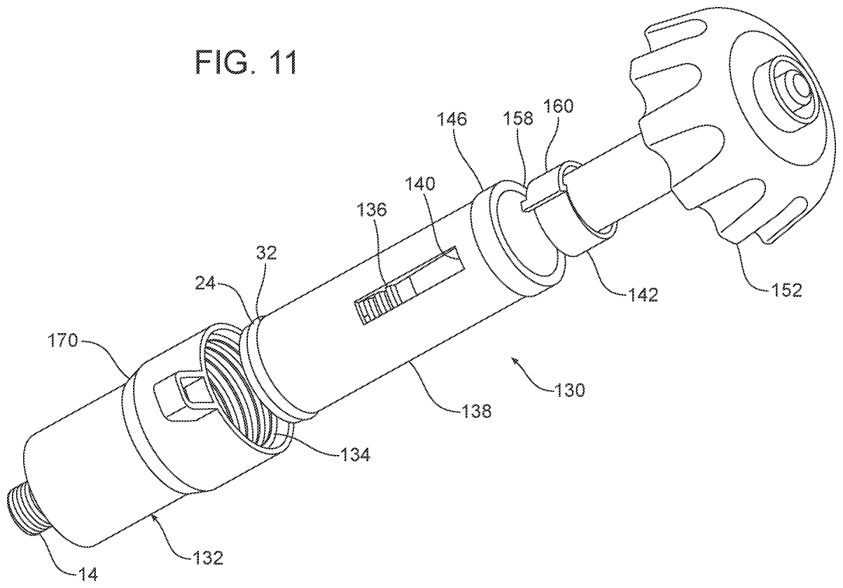

In another embodiment of a syringe 130 used for crushing medication(s), shown in FIG. 10, a barrel 132 of the syringe includes internal threads 134 for engaging retractable threads 136 of a plunger 138. This eliminates the need for the thread ring 80 of syringe 10. Otherwise, the plunger 138 is generally the same as the plunger described above in association with syringe 10 but the plunger tip is integrally molded and thus not removeable.

More specifically, the retractable threads 136 releasably engage with the internal threads 134 of the barrel 132. When engaged, the threads provide a similar mechanical advantage to generate a substantial force to advance the plunger 138 distally into the barrel 132 to crush medication(s). The retractable threads 136 are again integrally molded into the plunger 138 and pivot or move on a living hinge 140. During use, the retractable threads 136 are pushed outward for engagement with the barrel threads 134 by a cam 142 as in the syringe 10. Since the plunger tip is not removeable, the ejector 72 is not required in this embodiment. However, the plunger 138 includes a support ring, such as the support ring in syringe 10, that engages an axle 144 extending from the cam 142 which is positioned within an interior of the plunger 138. Even more, at least one rib, such as the rib 118 of syringe 10, provides mechanical strengthening and a stop point for rotation of the cam 142. In yet another alternate embodiment, the threads on the plunger 138 may be fixed for engagement with the barrel threads 134 when the plunger is inserted far enough into the barrel 132.

As in the syringe 10, a ring 146 is attached to or integrally molded with a proximal end of the plunger 138, and defines a ledge 148. One or more latches 150 extend from a handle 152 and are designed to engage the ring 146 and ledge 148 while allowing for rotation of the plunger 138 relative to the handle 152. Again, the latches 150 include deflectable arms 154 that extends from the handle 152 and include angled surfaces 156 which engage the ring 146 causing the arms to deflect outwardly when engaging and passing over the ring and to return to a normal, non-deflected, position in engagement with the ledge 148.

As noted above, the cam 142 has an axle 144 which engages a support ring 146 inside of the plunger 138 and is integrally molded to the handle 152. Essentially the handle 150, latches 148, and cam 142 are the same as in the syringe 10 including a stop 158 provided on a lobe 160 of the cam 142 to limit its rotation. At one limit of rotation, the retractable threads 136 are not engaged and are resting in their normal or retracted position. At the other limit of rotation, the retractable threads 136 are pushed outward at least sufficient to engage with the barrel threads 134.

The barrel 132 has distal and proximal portions 162, 164. The distal portion 162 is smooth and has a constant radius throughout its length. The plunger 138 with a gasket 166 or O-ring sealingly engages an interior wall of the distal portion 162 of the barrel 132. This sealing engagement functions the same as a medical syringe allowing the plunger 138 to force the contents of the chamber through an opening at a connector 168. Moving proximally, the proximal portion 164 of the barrel 132 has a larger diameter with threads 134 on its interior surface as shown in FIG. 12.

An interior radius of the threads 134 is slightly larger than an outside diameter of the plunger gasket 166 (or may be substantially equal to in other embodiments) so that the plunger 138 may pass through this portion without resistance from the threads. An intermediate portion 170 of the barrel 132 joins the inner diameters of the distal and proximal portions together, provides a transition with a guiding effect, and compresses the plunger gasket 166 as the plunger 138 is inserted and transitions through the intermediate portion. In alternate embodiments, the interior, distal surface of the barrel 132 may have ridges, creases, and/or contours to optimize crushing of the medication as described above with regard to syringe 10 and the alternate embodiments shown in FIG. 10. Grips 174 may be provided on an exterior surface of the barrel 132 which allow the user to stabilize the barrel both during a rotational motion or a linear motion as plunger 138 is either rotated or moved in or out of the barrel 132.

In use, the syringe 130 acts much the same as the syringe 10. The only differences revolve around the replacement of the thread ring 80 with the internal threads 134 of the barrel 132. Thus, a user removes the barrel 132 from the syringe 130 by rotating the handle 152 counterclockwise causing the cam 142 to disengage the retractable threads 136 allowing them to return to the normal, non-extended position. At this point, the plunger 138 is free to be pulled out of the barrel 132 in a linear manner. Medication to be crushed is placed into the chamber of the barrel 132. A cap or plug 172 may be placed to occlude the distal opening of the barrel 132 as needed. The barrel 132 is then reattached to the syringe 10.

More specifically, the plunger 138 is reinserted into the barrel 132. The user then rotates the handle 152 clockwise by gripping the grips 174 on the barrel 132 with one hand and the handle with the other hand. The rotation of the handle 152 activates the cam 142 to push the retractable threads 136 outward. At a certain point of rotation, the retractable threads 136 engage with the fixed threads 134 of the barrel 132, rotation of the cam 142 is stopped, and the plunger 138 is further advanced into the barrel 132 by the screw action. This provides the aforementioned mechanical advantage that generates the substantial force to crush the medication(s).

As described above, the medication(s) is crushed between the exterior surface of the plunger tip and the interior distal surface of the barrel 132 as the plunger is rotated and advanced within the barrel. When the medication is sufficiently crushed, the user rotates the handle 152 counterclockwise causing the cam 142 to disengage the retractable threads 136 allowing them to return to the normal, non-extended position. At this point, the plunger 138 is free to be pulled out of the barrel 132 in a linear manner. If the end cap or plug 172 has been used, the cap may now be removed.

In a next step, the distal fitting 168 of the barrel 132 may be dipped or positioned into a fluid suitable for mixing with the crushed medication(s) and injection into the body. The fluid may be sterile water contained in a cup (not shown) or other fluids with which the crushed medication(s) may be mixed. Once the barrel fitting 168 is positioned in the fluid, the plunger 138 is linearly withdrawn thereby pulling the fluid into the chamber of the barrel 132 where it mixes with the crushed medication(s). The barrel fitting 168 may then be attached to a feeding tube, via a corresponding fitting or otherwise, and the plunger 138 may be advanced to directly inject the mixture of crushed medication(s) and fluid into the feeding tube. If there are medication fragments remaining within the chamber of the barrel 132, additional fluid may be drawn into the chamber, and the injection process repeated.

As noted above, the barrel 132 of the syringe 130 is intended to be used for a single patient so that if any medication fragments remain within the barrel, they will not be inadvertently administered to a different patient. In an alternate embodiment, the entire syringe 130 may be intended to be used for a single patient and discarded after use. In order to replace the barrel 132, the user may simply remove the barrel 132 again (as described above) and properly discarded. A subsequent barrel may be positioned on the syringe 130 at this point or after subsequent medication(s) to be crushed is placed into the chamber of the subsequent barrel.

In addition, the word component as used herein refers to a body having internal threads. In the described embodiment, the component is the thread ring and in the alternate embodiment, the component forms a portion of the barrel. All such modifications and variations are within the scope of the appended claims when interpreted in accordance with the breadth to which they are fairly, legally and equitably entitled.

1-133. (canceled)

134. A syringe for crushing and injecting medication into a body, comprising:
a barrel having an inner diameter;
a plunger for longitudinal movement within the barrel, the plunger having threads along at least a portion thereof for engaging internal threads of a component; and
a handle for rotating the plunger relative the barrel.

135. The syringe for crushing and injecting medication into a body of claim 134,
wherein the component is a thread ring.

136. The syringe for crushing and injecting medication into a body of claim 134,
wherein a surface of a distal end of the plunger is hard.

137. The syringe for crushing and injecting medication into a body of claim 136, wherein the hard surface of the plunger engages an interior surface of the barrel to crush the medication.

138. The syringe for crushing and injecting medication into a body of claim 135, wherein the thread ring includes at least two arms extending along an exterior surface of the thread ring.

139. The syringe for crushing and injecting medication into a body of claim 134, wherein a ring defining a ledge is attached to a proximal end of the plunger, and wherein the handle includes at least one latch for engaging the ledge.

140. The syringe for crushing and injecting medication into a body of claim 134, wherein the threads of the plunger extend at least partially around the plunger and are retractable.

141. The syringe for crushing and injecting medication into a body of claim 134, wherein the threads are retractable and biased to a retracted position.

142. The syringe for crushing and injecting medication into a body of claim 134, further comprising a cam attached to the handle for selectively engaging the threads for movement between a retracted position and an extended position sufficient to engage the internal threads of the component.

143. The syringe for crushing and injecting medication into a body of claim 134, wherein the barrel includes a first portion, and a second portion including the component.

144. The syringe for crushing and injecting medication into a body of claim 143, wherein the hard surface of the plunger engages an interior surface of the barrel to crush the medication.

145. The syringe for crushing and injecting medication into a body of claim 143, wherein the threads of the plunger extend at least partially around the plunger and are retractable.

146. The syringe for crushing and injecting medication into a body of claim 143, wherein the threads are retractable and biased to a retracted position.

147. A syringe for crushing and injecting medication into a body, comprising:
a barrel;
a plunger for longitudinal movement within the barrel, wherein a surface of a distal end of the plunger is hard; and
a handle for rotating the plunger relative the barrel.

148. The syringe for crushing and injecting medication into a body of claim 147, wherein the hard surface of the plunger includes at least one of ridges or creases.

149. The syringe for crushing and injecting medication into a body of claim 148, wherein the interior surface of the barrel is a tapering wall having at least one of ridges or creases.

150. The syringe for crushing and injecting medication into a body of claim 147, wherein a ring defining a ledge is attached to a proximal end of the plunger, and wherein the handle includes at least one latch for engaging the ledge.

151. The syringe for crushing and injecting medication into a body of claim 147, further comprising a cam attached to the handle for selectively engaging threads of the plunger for movement between a retracted position and an extended position sufficient to engage internal threads of the component.

152. The syringe for crushing and injecting medication into a body of claim 147, wherein the plunger includes a detachable tip.

153. The syringe for crushing and injecting medication into a body of claim 152, wherein at least two locking tabs extend from the tip and engage a wall defining a proximal portion of a first receptacle to secure the tip to the plunger.

154. The syringe for crushing and injecting medication into a body of claim 147, further comprising an ejector.

155. The syringe for crushing and injecting medication into a body of claim 154, wherein a tip of an ejector is shaped to engage tapered surfaces of at least one of the at least two locking tabs and move the at least one of the at least two locking tabs from a normal position or intermediate position to a compressed position.

156. A syringe for crushing and injecting medication into a body, comprising:
a barrel having internal threads positioned along a first interior wall, a diameter of the internal threads being greater than or equal to a diameter of a second interior wall of the barrel; and
a plunger in sealing engagement with the second interior wall having threads for engaging the internal threads of the barrel.

157. The syringe for crushing and injecting medication into a body of claim 156, wherein a hard surface of a distal end of the plunger engages an interior surface of the barrel to crush the medication.

158. The syringe for crushing and injecting medication into a body of claim 157 wherein the interior surface is a tapering frustroconical shaped wall having radially directed creases.

159. The syringe for crushing and injecting medication into a body of claim 156, wherein a ring defining a ledge is attached to a proximal end of the plunger, and wherein the handle includes at least one latch for engaging the ledge.

160. The syringe for crushing and injecting medication into a body of claim 156, wherein the threads are biased to a retracted position.

161. The syringe for crushing and injecting medication into a body of claim 156, further comprising a cam attached to the handle for selectively engaging the threads for movement between a retracted position and an extended position sufficient to engage the internal threads of the barrel.
